(19) United States
(12) Patent Application Publication
(10) Pub. No.: US 2024/0266042 A1
(43) Pub. Date: Aug. 8, 2024

(54) TERMINAL CONTROLLING APPARATUS, TERMINAL CONTROLLING METHOD, AND NON-TRANSITORY COMPUTER-READABLE STORAGE MEDIUM

(71) Applicant: CANON MEDICAL SYSTEMS CORPORATION, Tochigi (JP)

(72) Inventor: Mikihito HAYASHI, Otawara (JP)

(73) Assignee: CANON MEDICAL SYSTEMS CORPORATION, Tochigi (JP)

(21) Appl. No.: 18/432,593

(22) Filed: Feb. 5, 2024

(30) Foreign Application Priority Data
Feb. 7, 2023 (JP) 2023-016765

(51) Int. Cl. G16H 40/63; G06T 7/70; G06V 40/10; A61B 90/00

(52) U.S. Cl. CPC G16H 40/63; G06T 7/70; G06V 40/10; A61B 90/08; G06T 2207/30004

(57) ABSTRACT

A terminal controlling apparatus according to an embodiment is a terminal controlling apparatus to control a terminal apparatus used by an operator of a medical machine and includes processing circuitry. The processing circuitry is configured to identify the position of the terminal apparatus. The processing circuitry is configured to judge, on the basis of the position of the terminal apparatus, whether or not safety is ensured about a specific operation to control driving of the medical machine. When it is determined that the safety of the operation is ensured, the processing circuitry is configured to control the terminal apparatus so as to become able to receive the operation from the operator.

### CROSS-REFERENCE TO RELATED APPLICATIONS

This application is based upon and claims the benefit of priority from Japanese Patent Application No. 2023-016765, filed on Feb. 7, 2023; the entire contents of which are incorporated herein by reference.

### FIELD

Embodiments described herein relate generally to a terminal controlling apparatus, a terminal controlling method, and a non-transitory computer-readable storage medium.

### BACKGROUND

Conventionally, medical facilities such as hospitals and clinics use various types of medical machines for imaging examined subjects, such as X-ray Computed Tomography (CT) apparatuses, X-ray diagnosis apparatuses, and Magnetic Resonance Imaging (MRI) apparatuses. Those medical machines are configured to receive, from operators, various types of operations such as operations related to the imaging and operations related to processing the images and are configured to work in accordance with the received operations.

In relation to the above, among the operations related to the medical machines, for example, specific operations to control driving of the medical machines (e.g., an operation to control X-ray emissions and an operation to control moving of a table/couch on which an examined subject is placed) are often performed by using a dedicated operation machine directly linked to each medical machine, primarily from a safety viewpoint.

As other examples, in recent years, systems have been considered in which various types of medical information related to diagnoses/treatment and medical examinations are provided for operators of a medical machine, by connecting, via a network, the medical machine configured to image an examined subject, a processing apparatus configured to perform various types of processes by using data acquired by the medical machine, and a terminal apparatus used by an operator.

In such systems, as explained above, when the medical machine for imaging the examined subject includes the dedicated operation machine for performing the specific operations to control the driving of the medical machine, the operator of the medical machine is required to operate the two types of machines, namely, the operation machine and the terminal apparatus for using the system. Thus, there is a concern that the operations related to the medical machine may become cumbersome.

### BRIEF DESCRIPTION OF THE DRAWINGS

FIG. 1 is a diagram illustrating an exemplary configuration of a medical information processing system according to a first embodiment;

FIG. 2 is a diagram illustrating an exemplary configuration of an X-ray CT apparatus according to the first embodiment;

FIG. 3 is a diagram illustrating an exemplary configuration of a medical information processing apparatus according to the first embodiment;

FIG. 4 is a flowchart illustrating a processing procedure of processes performed by processing functions included in processing circuitry according to the first embodiment;

FIG. 5 is a diagram illustrating exemplary configurations of an examination room and an operation room where the X-ray CT apparatus according to the first embodiment is installed;

FIG. 6 is a drawing illustrating an example of an operation screen displayed on a terminal apparatus by a controlling function according to the first embodiment;

FIG. 7 is a flowchart illustrating a processing procedure of processes performed by processing functions included in processing circuitry according to a second embodiment; and

FIG. 8 is a flowchart illustrating a processing procedure of processes performed by processing functions included in processing circuitry according to a third embodiment.

### DETAILED DESCRIPTION

A terminal controlling apparatus according to an embodiment is a terminal controlling apparatus to control a terminal apparatus used by an operator of a medical machine and includes an identifying unit, a judging unit, and a controlling unit. The identifying unit is configured to identify the position of the terminal apparatus. The judging unit is configured to judge, on the basis of the position of the terminal apparatus, whether or not safety is ensured about a specific operation to control driving of the medical machine. When it is determined that the safety of the operation is ensured, the controlling unit is configured to control the terminal apparatus so as to become able to receive the operation from the operator.

Exemplary embodiments of a terminal controlling apparatus, a terminal controlling method, and a non-transitory computer-readable storage medium will be explained in detail, with reference to the accompanying drawings. In the following embodiments, examples will be explained in which a terminal controlling apparatus, a terminal controlling method, and a terminal controlling program of the present disclosure are applied to a medical information processing apparatus included in a medical information processing system configured to provide various types of medical information related to diagnoses/treatment and/or medical examinations for an examined subject, in response to a request from an operator.

### First Embodiment

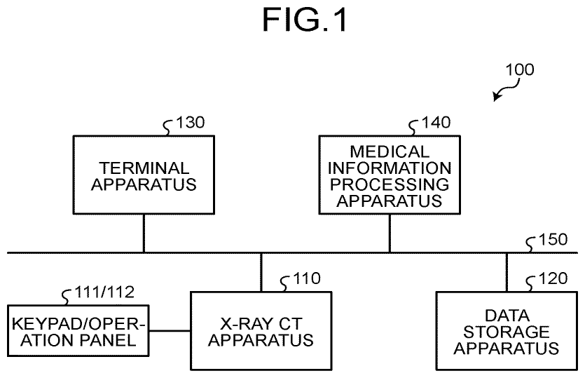

FIG. 1 is a diagram illustrating an exemplary configuration of a medical information processing system 100 according to a first embodiment.

As illustrated in FIG. 1, for example, the medical information processing system 100 includes an X-ray CT apparatus 110, a data storage apparatus 120, a terminal apparatus 130, and a medical information processing apparatus 140. In this situation, the apparatuses are installed in a medical facility such as a hospital or a clinic, and are connected so as to be able to communicate with one another via a network 150 such as an intra-hospital Local Area Network (LAN). The X-ray CT apparatus 110 is an example of a medical machine.

The X-ray CT apparatus 110 is configured to obtain CT images of an examined subject (hereinafter “patient”). More specifically, the X-ray CT apparatus 110 is configured to acquire projection data indicating a distribution of X-rays that have passed through the patient, by causing an X-ray tube and an X-ray detector to rotate and move on a circular trajectory surrounding the patient. Further, the X-ray CT apparatus 110 is configured to generate image data of the CT images by performing an image processing process such as a reconstructing process on the acquired projection data. Also, the X-ray CT apparatus 110 is configured to save the projection data and the image data in the data storage apparatus 120, by transmitting the acquired projection data and the generated image data to the data storage apparatus 120 via the network 150.

The data storage apparatus 120 is configured to save, in a storage medium provided therein, the projection data acquired by the X-ray CT apparatus 110, the image data generated by the X-ray CT apparatus 110, and the like. For example, the data storage apparatus 120 is realized by using a storage apparatus such as a hard disk or an optical disk, or a file server or the like.

The terminal apparatus 130 is used by an operator of the X-ray CT apparatus 110 and is configured, in response to an instruction from the operator, to request various types of medical information related to diagnoses/treatment and/or medical examinations for the patient from the medical information processing apparatus 140. Further, the terminal apparatus 130 is configured to cause a display to display the medical information transmitted thereto from the medical information processing apparatus 140 in response to the request. For example, the terminal apparatus 130 is realized by using a portable computer machine such as a tablet terminal or a laptop personal computer.

The medical information processing apparatus 140 is configured to generate the various types of medical information related to the diagnoses/treatment and/or the medical examinations for the patient, by performing various types of processes in response to the request transmitted from the terminal apparatus 130, and is configured to transmit the generated medical information to the terminal apparatus 130. For example, the medical information processing apparatus 140 is realized by using a computer machine such as a server, a workstation, or a personal computer or by using a plurality of computer machines configured to work in collaboration with one another as a single computer system.

An overall configuration of the medical information processing system 100 according to the present embodiment has thus been explained. In the medical information processing system 100, the X-ray CT apparatus 110 includes, in the present embodiment, dedicated operation machines for performing specific operations to control driving of the X-ray CT apparatus 110.

In the present example, the specific operation to control the driving of the X-ray CT apparatus 110 denotes a certain operation having a possibility, when the X-ray CT apparatus 110 starts working as a result of the operation, of affecting, from a safety viewpoint, the operator or someone else other than the operator being present in the vicinity of the X-ray CT apparatus 110, the operation requiring that safety be ensured, before being performed, for the operator or someone else who is present in the vicinity of the X-ray CT apparatus 110.

For instance, concrete examples of the specific operation include an operation to control X-ray emissions and an operation to control moving of the table. In the operation to control the X-ray emissions, if the operator or someone else is in an X-ray emission range, there is a possibility that the operator or someone else may be exposed to radiation. Further, in the operation to control moving of the table, if the operator or someone else is in the vicinity of the table, there is a possibility that the table may interfere with the operator or someone else.

In the present embodiment, primarily from a safety viewpoint, such specific operations are performed by using dedicated operation machines directly linked to the X-ray CT apparatus 110. In the following sections, an example will be explained in which, as the dedicated operation machines, the X-ray CT apparatus 110 includes a keypad 111 for performing the operation to control the X-ray emissions and an operation panel 112 for performing the operation to control the moving of the table.

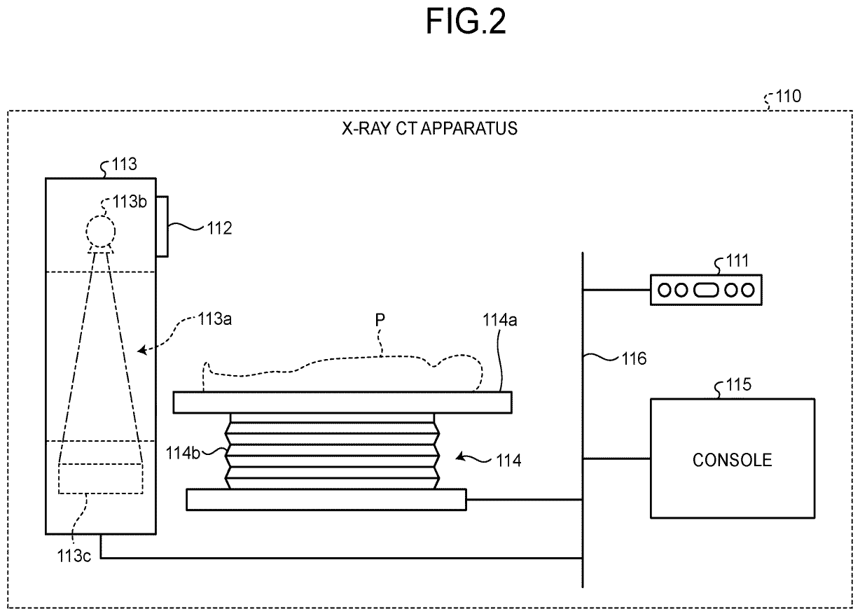

FIG. 2 is a diagram illustrating an exemplary configuration of the X-ray CT apparatus 110 according to the first embodiment.

As illustrated in FIG. 2, for example, the X-ray CT apparatus 110 includes a gantry 113, a table 114, a console 115, the keypad 111, and the operation panel 112.

The gantry 113 is an apparatus configured to acquire projection data of an examined subject (e.g., a patient) P. More specifically, the gantry 113 includes an X-ray tube 113b and an X-ray detector 113c arranged so as to oppose each other while an opening part 113a having a circular cylindrical shape is interposed therebetween. The gantry 113 is configured to acquire the projection data indicating a distribution of X-rays that have passed through the patient P, under an imaging condition received by a console 125, by causing the X-ray tube 113b and the X-ray detector 113c to rotate and move on a circular trajectory surrounding the patient P arranged in the opening part 113a. In this situation, the gantry 113 is configured to start or stop the X-ray emissions in response to an instruction from the operator, by controlling the X-ray tube 113b on the basis of a control signal transmitted thereto from the keypad 111 (explained later).

The table 114 is an apparatus on which, at the time of imaging, the patient P is placed. More specifically, the table 114 includes a tabletop 114a on which the patient P is placed and a table base part 114b configured to movably support the tabletop 114a. As a result of causing a drive apparatus provided for the table base part 114b to work, the table 114 is configured to move the tabletop 114a in up-and-down directions, left-and-right directions, and front-and-back directions, with respect to the gantry 113. For example, when the patient P is to be imaged, the table 114 is configured to move the tabletop 114a on which the patient P is placed, to an imaging position within the opening part 113a of the gantry 113. In this situation, as a result of controlling the drive apparatus in the table base part 114b on the basis of a control signal transmitted from the operation panel 112 (explained later), the table 114 is configured to move the tabletop 114a in various directions, in response to instructions from the operator.

The console 125 is configured to receive various types of operations related to the X-ray CT apparatus 110 from the operator and to control working of functional units of the X-ray CT apparatus 110 in response to the received operations. For example, the console 125 is configured to receive, from the operator, the imaging condition used at the time of acquiring the projection data of the patient P, a reconstruction condition used at the time of generating the image data of the CT images from the projection data by performing the reconstructing process, an image processing condition used at the time of generating image data of a post-processed image from the CT images by performing a post-processing process, and the like. Further, the console 125 is configured to acquire the projection data of the patient P, by controlling functional units of the gantry 113 on the basis of the imaging condition received from the operator. Also, the console 125 is configured to generate the image data of the CT images by performing the reconstructing process on the projection data acquired by the gantry 113, on the basis of the reconstruction condition received from the operator. In addition, the console 125 is configured to generate the image data of the post-processed image, by performing the post-processing process on the image data of the CT images on the basis of the image processing condition received from the operator. Furthermore, the console 125 is configured to save the projection data and the image data in the data storage apparatus 120, by transmitting the acquired projection data and the generated image data to the data storage apparatus 120 via the network 150.

The keypad 111 is configured to receive, from the operator, the operation to control the X-ray emissions performed by the gantry 113. More specifically, the keypad 111 is configured to receive, from the operator, an operation to start the X-ray emissions, an operation to stop the X-ray emissions, and the like. In this situation, the keypad 111 is connected so as to be directly linked to the gantry 113, via a bus 116 or the like inside the X-ray CT apparatus 110 and is configured to convert the operation received from the operator into a control signal and to transmit the control signal to the gantry 113.

The operation panel 112 is configured to receive, from the operator, the operation to control the moving of the table 114. More specifically, the operation panel 112 is configured to receive, from the operator, an operation to move the tabletop 114a in an up-and-down direction, an operation to move the same in a left-and-right direction, an operation to move the same in a front-and-back direction, and the like. In this situation, the operation panel 112 is provided for the gantry 113, is connected so as to be directly linked to the table 114 via the gantry 113 and the bus 116, and is configured to convert the operation received from the operator into a control signal and to transmit the control signal to the table 114. As explained above, when the X-ray CT apparatus 110 includes the dedicated operation machines (the keypad 111 and the operation panel 112) for performing the specific operations to control the driving of the X-ray CT apparatus 110, the operator of the X-ray CT apparatus 110 is required to operate the two types of machines, namely, the operation machines and the terminal apparatus 130 for using the medical information processing system 100. Thus, there is a concern that the operations related to the X-ray CT apparatus 110 may become cumbersome.

For this reason, in the present embodiment, the medical information processing apparatus 140 is configured to control the terminal apparatus 130 used by the operator of the X-ray CT apparatus 110, to make it possible to conveniently perform the operations related to the X-ray CT apparatus 110.

More specifically, the medical information processing apparatus 140 makes it possible to conveniently perform the operations related to the X-ray CT apparatus 110, by additionally enabling the terminal apparatus 130 to perform the certain operations that are performed by using the dedicated operation machines directly linked to the X-ray CT apparatus 110 primarily from a safety viewpoint.

Next, a configuration of the medical information processing apparatus 140 according to the present embodiment will be explained in detail.

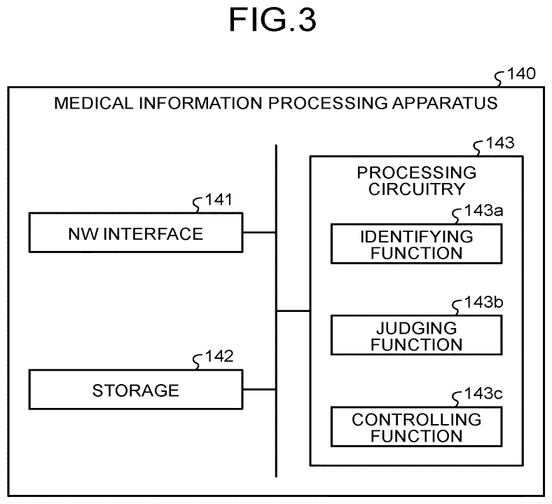

FIG. 3 is a diagram illustrating an exemplary configuration of the medical information processing apparatus 140 according to the first embodiment.

As illustrated in FIG. 3, for example, the medical information processing apparatus 140 includes a network (NW) interface 141, a storage 142, and processing circuitry 143.

The NW interface 141 is configured to control transfer of various types of data and communication performed between the medical information processing apparatus 140 and other apparatuses connected via the network 150. More specifically, the NW interface 141 is connected to the processing circuitry 143 and is configured, under control of the processing circuitry 143, to control the transfer of the various types of data and the communication. For example, the NW interface 141 is realized by using a network card, a network adaptor, a Network Interface Controller (NIC), or the like.

The storage 142 is configured to store therein various types of data, various types of programs, and the like. More specifically, the storage 142 is connected to the processing circuitry 143 and is configured, under the control of the processing circuitry 143, to store therein and read therefrom the various types of data and the various types of programs. For example, the storage 142 is realized by using a semiconductor memory element such as a Random Access Memory (RAM) or a flash memory, or a hard disk, an optical disk, or the like.

The processing circuitry 143 is configured to control constituent elements of the medical information processing apparatus 140 in accordance with input operations received from the operator via the NW interface 141. For example, the processing circuitry 143 is configured to store the data received by the NW interface 141, into the storage 142.

With the configurations described above, in the present embodiment, by performing the various types of processes in response to the requests transmitted from the terminal apparatus 130 used by the operator of the X-ray CT apparatus 110, the processing circuitry 143 is configured to generate the various types of medical information related to the diagnoses/treatment and/or the medical examinations for the patient and is configured to further transmit the generated medical information to the terminal apparatus 130.

For example, the processing circuitry 143 is configured to receive, from the terminal apparatus 130, the imaging condition used at the time of acquiring the projection data of the patient P, the reconstruction condition used at the time of generating the image data of the CT images from the projection data by performing the reconstructing process, the image processing condition used at the time of generating the image data of the post-processed image from the CT images by performing the post-processing process, and the like. Further, the processing circuitry 143 is configured to acquire the projection data of the patient P, by controlling functional units in the gantry 113 of the X-ray CT apparatus 110, on the basis of the imaging condition received from the terminal apparatus 130. In addition, the processing circuitry 143 is configured to generate the image data of the CT images by performing the reconstructing process on the projection data saved in the data storage apparatus 120 and to further transmit the generated image data to the terminal apparatus 130. Also, the processing circuitry 143 is configured to generate the image data of the post-processed image by performing the post-processing process on the image data of the CT images saved in the data storage apparatus 120 and to further transmit the generated image data to the terminal apparatus 130.

Further, for example, the processing circuitry 143 is configured to generate an analysis result of the image data, by performing various types of analyzing processes on the image data saved in the data storage apparatus 120 and to further transmit the generated analysis result to the terminal apparatus 130. In addition, for example, the processing circuitry 143 is configured to generate an image interpretation report for the patient, on the basis of the image data saved in the data storage apparatus 120 and diagnosis information or the like input by the operator and to further transmit the generated image interpretation report to the terminal apparatus 130.

Furthermore, for example, the processing circuitry 143 is configured to receive, via the network 150, a medical examination order from a Hospital Information System (HIS), a Radiology Information System (RIS) or the like installed in the medical facility, to further generate a plurality of workflow candidates related to a diagnosis/treatment process or a medical examination for the patient on the basis of the received medical examination order, and to transmit the generated workflow candidates to the terminal apparatus 130.

In addition, in the present embodiment, the processing circuitry 143 further has a processing function for controlling the terminal apparatus 130, so as to additionally enable the terminal apparatus 130 to perform the certain operations that are performed by using the keypad 111 and the operation panel 112 directly linked to the X-ray CT apparatus 110, i.e., the operation to control the X-ray emissions and the operation to control the moving of the table 114.

More specifically, as illustrated in FIG. 3, the processing circuitry 143 includes an identifying function 143a, a judging function 143b, and a controlling function 143c. In this situation, the identifying function 143a is an example of the identifying unit. The judging function 143b is an example of the judging unit. The controlling function 143c is an example of the controlling unit.

The identifying function 143a is configured to identify the position of the terminal apparatus 130 and a state of the operator.

Further, on the basis of the position of the terminal apparatus 130 and the state of the operator identified by the identifying function 143a, the judging function 143b is configured to judge whether or not safety is ensured about the operation to control the X-ray emissions and the operation to control the moving of the table 114.

Further, when the judging function 143b determines that the safety is ensured about the operation to control the X-ray emissions and the operation to control the moving of the table 114, the controlling function 143c is configured to control the terminal apparatus 130 so as to become able to receive the operations from the operator of the X-ray CT apparatus 110.

In this situation, the safety of the operation to control the X-ray emissions and the operation to control the moving of the table 114 being ensured denotes that the terminal apparatus 130 is in a certain position that allows the X-ray CT apparatus 110 to perform the X-ray emissions and to move the table 114 safely, while the operator is in a certain state that allows the X-ray CT apparatus 110 to perform the X-ray emissions and to move the table 114 safely.

In the present embodiment, the processing circuitry 143 is realized by using one or more processors, for example. In that situation, the processing functions described above are stored in the storage 142 in the form of computer-executable programs. Further, the processing circuitry 143 is configured to realize the functions corresponding to the programs, by reading and executing the programs stored in the storage 142. In other words, the processing circuitry 143 that has read the programs has the processing functions illustrated in FIG. 3.

Next, processes performed by the processing functions included the processing circuitry 143 according to the present embodiment will be explained in detail.

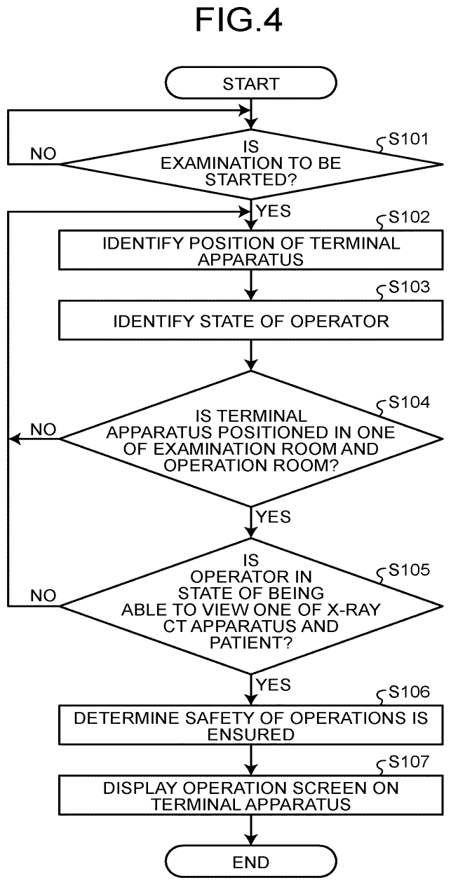

FIG. 4 is a flowchart illustrating a processing procedure of the processes performed by the processing functions included in the processing circuitry 143 according to the first embodiment.

As illustrated in FIG. 4, in the present embodiment, to begin with, the identifying function 143a receives an instruction to start a medical examination, from the terminal apparatus 130 (step S101).

For example, in response to an instruction from the operator, the identifying function 143a may be configured to receive, from the terminal apparatus 130, information related to the patient to be imaged and to also receive the instruction to start the medical examination together with the information. Alternatively, for example, in response to an instruction from the operator, the identifying function 143a may be configured to receive, from the terminal apparatus 130, an instruction to select a workflow from among the plurality of workflow candidates described above and to also receive the instruction to start the medical examination together with the instruction.

In this situation, in the present embodiment, the X-ray CT apparatus 110 is installed while being separated in the two rooms, namely, the examination room and the operation room. Further, in the present embodiment, the terminal apparatus 130 is configured to be able to communicate with the medical information processing apparatus 140, by using a wireless communication technique such as WiFi (registered trademark) or Bluetooth (registered trademark).

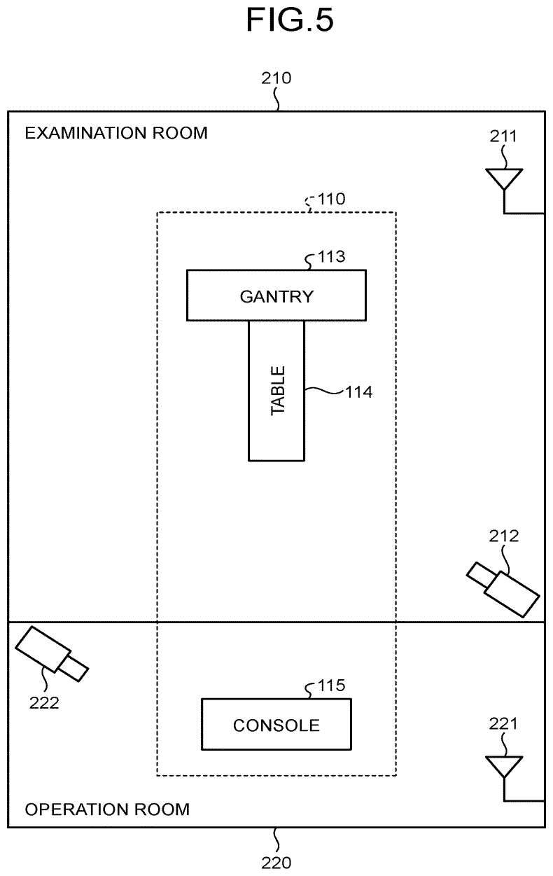

FIG. 5 is a diagram illustrating exemplary configurations of an examination room 210 and an operation room 220 where the X-ray CT apparatus 110 according to the first embodiment is installed.

As illustrated in FIG. 5, for example, of the X-ray CT apparatus 110, the gantry 113 and the table 114 are installed in the examination room 210, whereas the console 115 is installed in the operation room 220. In this situation, the examination room 210 is provided with: an antenna 211 used by the terminal apparatus 130 for wirelessly communicating with the medical information processing apparatus 140; and a camera 212 for monitoring the inside of the examination room 210. Similarly, the operation room 220 is also provided with: an antenna 221 used by the terminal apparatus 130 for wirelessly communicating with the medical information processing apparatus 140; and a camera 222 for monitoring the inside of the operation room 220.

Subsequently, upon receipt of the instruction to start the medical examination from the terminal apparatus 130 (step S101: Yes), the identifying function 143a identifies the position of the terminal apparatus 130, by using a wireless communication technique such as WiFi (registered trademark) or Bluetooth (registered trademark) (step S102).

In addition, the identifying function 143a identifies a state of the operator, by using an image obtained by the camera 212 in the examination room 210 or by the camera 222 in the operation room 220 where the X-ray CT apparatus 110 is installed (step S103).

Subsequently, on the basis of the position of the terminal apparatus 130 identified by the identifying function 143a, the judging function 143b judges whether or not the terminal apparatus 130 is positioned in one of the examination room 210 and the operation room 220 (step S104).

Further, when the terminal apparatus 130 is positioned in one of the examination room 210 and the operation room 220 (step S104: Yes), the judging function 143b judges whether or not the operator is in a state of being able to view one of the X-ray CT apparatus 110 and the patient, on the basis of the state of the operator identified by the identifying function 143a (step S105).

For example, the judging function 143b determines the direction of the line of sight of the operator, on the basis of an image obtained by the camera 212 in the examination room 210 or by the camera 222 in the operation room 220. When the line of sight of the operator is directed toward one of the X-ray CT apparatus 110 and the patient, the judging function 143b determines that the operator is in the state of being able to view one of the X-ray CT apparatus 110 and the patient.

After that, when the operator is in the state of being able to view one of the X-ray CT apparatus 110 and the patient (step S105: Yes), the judging function 143b determines that the safety is ensured about the operation to control the X-ray emissions and the operation to control the moving of the table 114 (step S106).

Subsequently, when the judging function 143b determines that the safety is ensured about the operation to control the X-ray emissions and the operation to control the moving of the table 114 (step S106), the controlling function 143c causes the terminal apparatus 130 to display an operation screen for receiving the operations from the operator (step S107).

As a result, the terminal apparatus 130 is enabled to receive, from the operator, the operation to control the X-ray emissions and the operation to control the moving of the table 114.

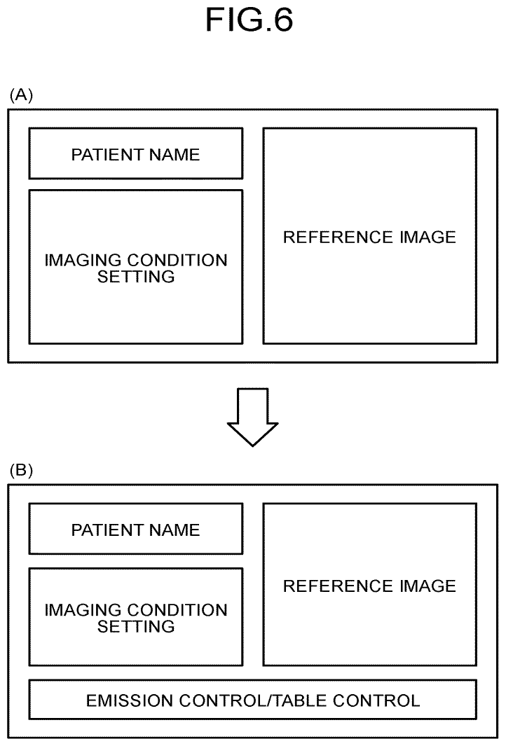

FIG. 6 is a drawing illustrating an example of the operation screen displayed on the terminal apparatus 130 by the controlling function 143c according to the first embodiment.

As illustrated in a portion (A) of FIG. 6, for example, at normal times, the controlling function 143c causes the terminal apparatus 130 to display a screen (PATIENT NAME) for receiving an operation to input the patient name of the patient being the examined subject, a screen (IMAGING CONDITION SETTING) for receiving an operation to set an imaging condition, and a screen (REFERENCE IMAGE) for displaying a reference image.

Further, as illustrated in a portion (B) of FIG. 6, for example, when it is determined that the safety is ensured about the operation to control the X-ray emissions and the operation to control the moving of the table 114, the controlling function 143c causes the terminal apparatus 130 to display, in addition to the three screens displayed at normal times, an operation screen (EMISSION CONTROL/TABLE CONTROL) for receiving the operation to control the X-ray emissions and the operation to control the moving of the table 114.

Further, as explained above, when the processing circuitry 143 is realized by using one or more processors, the processes at steps S101 through S103 described above are realized as a result of the processing circuitry 143 reading and executing the program corresponding to the identifying function 143a from the storage 142, for example. Further, the processes at steps S104 and S105 described above are realized as a result of the processing circuitry 143 reading and executing the program corresponding to the judging function 143b from the storage 142, for example. Also, the process at step S106 is realized as a result of the processing circuitry 143 reading and executing the program corresponding to the controlling function 143c from the storage 142, for example.

As explained above, in the first embodiment, the identifying function 143a is configured to identify the position of the terminal apparatus 130 and the state of the operator. Further, on the basis of the position of the terminal apparatus 130 and the state of the operator identified by the identifying function 143a, the judging function 143b is configured to judge whether or not the safety is ensured about the operation to control the X-ray emissions and the operation to control the moving of the table 114. Further, when the judging function 143b determines that the safety is ensured about the operation to control the X-ray emissions and the operation to control the moving of the table 114, the controlling function 143c is configured to control the terminal apparatus 130 so as to become able to receive the operations from the operator of the X-ray CT apparatus 110.

With the configurations above, because the safety is ensured about the operation to control the X-ray emissions and the operation to control the moving of the table 114, the terminal apparatus 130 for general use is additionally enabled to perform the certain operations that are performed by using the dedicated keypad 111 and operation panel 112, primarily from a safety viewpoint. Consequently, according to the first embodiment, it is possible to conveniently perform the operations related to the X-ray CT apparatus 110. In addition, because the operations related to the X-ray CT apparatus 110 become convenient, it is possible to quickly perform the medical examination for the patient.

Further, with the configurations described above, because the terminal apparatus 130 for general use becomes able to perform the certain operations that are performed by using the dedicated keypad 111 and operation panel 112, it is also possible to eliminate the keypad 111 and the operation panel 112 from the constituent elements of the X-ray CT apparatus 110. As a result, it is possible to reduce costs of the X-ray CT apparatus 110.

Further, in the first embodiment, the terminal apparatus 130 is configured to be able to communicate with the medical information processing apparatus 140 by using the wireless communication technique. The identifying function 143a is configured to identify the position of the terminal apparatus 130 by using the wireless communication technique. When the terminal apparatus 130 is positioned inside the rooms where the X-ray CT apparatus 110 is installed, while the operator is in the state of being able to view one of the X-ray CT apparatus 110 and the patient, the judging function 143b is configured to determine that the safety is ensured about the operation to control the X-ray emissions and the operation to control the moving of the table 114.

With the configurations described above, when the terminal apparatus 130 is used inside the rooms where the X-ray CT apparatus 110 is installed, the terminal apparatus 130 is additionally enabled to perform the certain operations that are performed by using the dedicated keypad 111 and operation panel 112.

The first embodiment has thus been explained. Further, it is also possible to carry out the above embodiment, by changing, as appropriate, the configuration of any of the apparatuses included in the medical information processing system 100. Thus, in the following sections, modification examples of the first embodiment will be explained as other embodiments. In the following embodiments, differences from the first embodiment will primarily be explained. Detailed explanations of duplicate features will be omitted. Second Embodiment

For example, in the first embodiment, the example was explained in which the position of the terminal apparatus 130 is identified by using the wireless communication technique; however, possible embodiments are not limited to this example. For instance, it is also acceptable to provide the X-ray CT apparatus 110 with a connection part to which the terminal apparatus 130 can be connected, so that the position of the terminal apparatus 130 is identified by detecting that the terminal apparatus 130 is connected to the connection part. In the following sections, an example with this configuration will be explained as a second embodiment.

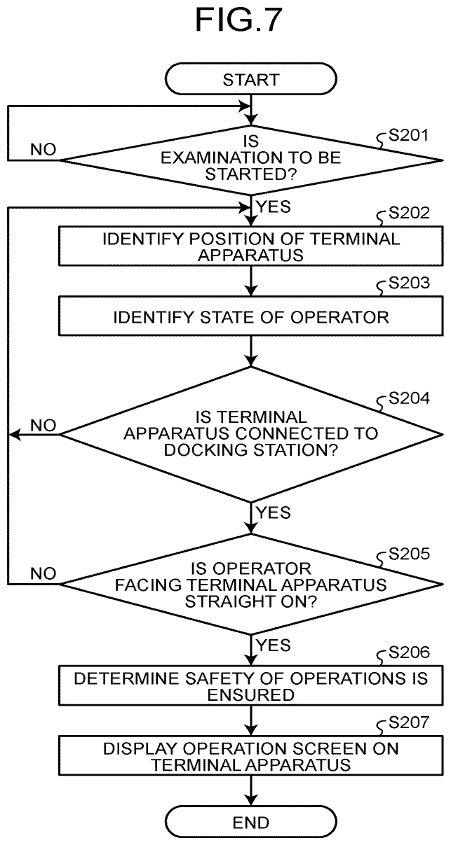

FIG. 7 is a flowchart illustrating a processing procedure of processes performed by the processing functions included in the processing circuitry 143 according to the second embodiment.

As illustrated in FIG. 7, in the present embodiment, to begin with, the identifying function 143a receives an instruction to start a medical examination, from the terminal apparatus 130, similarly to the first embodiment (step S201).

In this situation, in the present embodiment, the X-ray CT apparatus 110 includes a docking station to which the terminal apparatus 130 can be connected. The docking station is an example of the connection part. For example, the docking station may be provided for the gantry 113 of the X-ray CT apparatus 110 or may be provided for the console 115. In addition, in the present embodiment, the terminal apparatus 130 includes a camera for imaging the operator.

Further, upon receipt of the instruction to start the medical examination from the terminal apparatus 130 (step S201: Yes), the identifying function 143a identifies the position of the terminal apparatus 130, by detecting that the terminal apparatus 130 is connected to the docking station of the X-ray CT apparatus 110 (step S202).

In addition, the identifying function 143a identifies a state of the operator, by using an image obtained by the camera 212 in the examination room 210 or by the camera 222 in the operation room 220 where the X-ray CT apparatus 110 is installed or an image obtained by the camera of the terminal apparatus 130 (step S203).

Subsequently, on the basis of the position of the terminal apparatus 130 identified by the identifying function 143a, the judging function 143b judges whether or not the terminal apparatus 130 is connected to the docking station of the X-ray CT apparatus 110 (step S204).

In addition, when the terminal apparatus 130 is connected to the docking station (step S204: Yes), the judging function 143b judges whether or not the operator is facing the terminal apparatus 130 straight on, on the basis of the state of the operator identified by the identifying function 143a (step S205).

For example, the judging function 143b determines the orientation of the body of the operator, on the basis of an image obtained by the camera 212 in the examination room 210 or by the camera 222 in the operation room 220, or an image obtained by the camera of the terminal apparatus 130. When the body of the operator is oriented toward the terminal apparatus 130, the judging function 143b determines that the operator is facing the terminal apparatus 130 straight on.

Further, when the operator is facing the terminal apparatus 130 straight on (step S205: Yes), the judging function 143b determines that the safety is ensured about the operation to control the X-ray emissions and the operation to control the moving of the table 114 (step S206).

Subsequently, when the judging function 143b determines that the safety is ensured about the operation to control the X-ray emissions and the operation to control the moving of the table 114 (step S206), the controlling function 143c causes the terminal apparatus 130 to display an operation screen for receiving the operations from the operator, similarly to the first embodiment (step S207).

As a result, the terminal apparatus 130 is enabled to receive, from the operator, the operation to control the X-ray emissions and the operation to control the moving of the table 114.

As explained above, in the second embodiment, the X-ray CT apparatus 110 includes the connection part to which the terminal apparatus 130 can be connected. The identifying function 143a is configured to identify the position of the terminal apparatus 130 by detecting that the terminal apparatus 130 is connected to the connection part of the X-ray CT apparatus 110. Further, the judging function 143b is configured to determine that the safety is ensured about the operation to control the X-ray emissions and the operation to control the moving of the table 114, when the terminal apparatus 130 is connected to the connection part of the X-ray CT apparatus 110, while the operator is facing the terminal apparatus 130 straight on.

With the configurations described above, even when it is not possible to perform wireless communication in the rooms where the X-ray CT apparatus 110 is installed, the terminal apparatus 130 can additionally be enabled to perform the certain operations that are performed by using the dedicated keypad 111 and operation panel 112.

### Third Embodiment

Further, for example, in the first embodiment, the example was explained in which the terminal apparatus 130 is used inside the rooms where the X-ray CT apparatus 110 is installed; however, possible embodiments are not limited to this example. For instance, the terminal apparatus 130 may be used in a predetermined location other than the rooms where the X-ray CT apparatus 110 is installed. In the following sections, this example will be explained as a third embodiment.

The following will describe in detail processes performed by the processing functions included in the processing circuitry 143 according to the present embodiment.

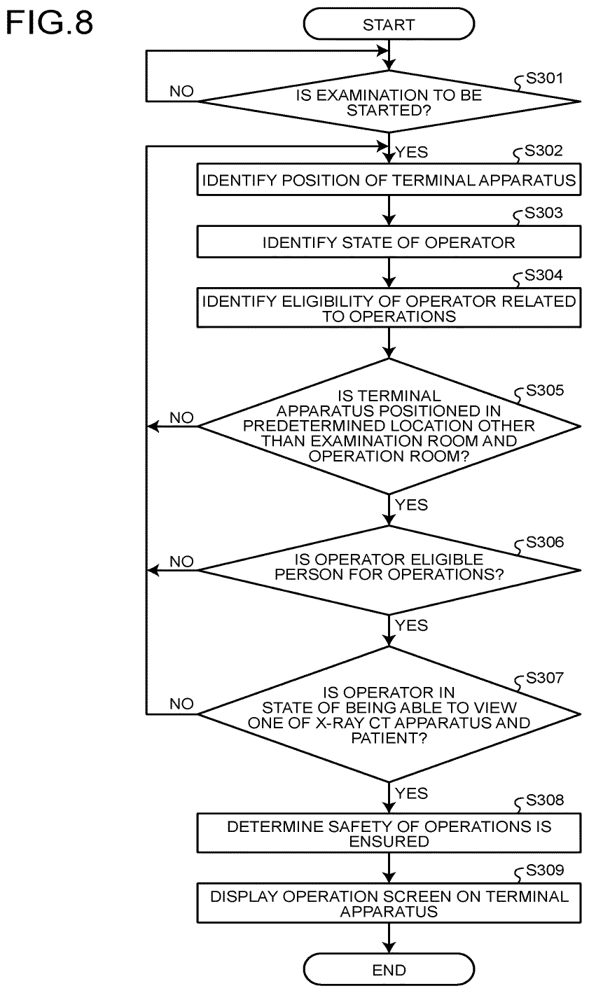

FIG. 8 is a flowchart illustrating the processing procedure of the processes performed by the processing functions included in the processing circuitry 143 according to the third embodiment.

As illustrated in FIG. 8, in the present embodiment, to begin with, the identifying function 143a receives an instruction to start a medical examination, from the terminal apparatus 130, similarly to the first embodiment (step S301).

In this situation, in the present embodiment, the terminal apparatus 130 is configured to be able to communicate with the medical information processing apparatus 140, by using a wireless communication technique such as WiFi (registered trademark) or Bluetooth (registered trademark), similarly to the first embodiment. Also, the terminal apparatus 130 includes a camera for imaging the operator, similarly to the second embodiment. In addition, in the present embodiment, the terminal apparatus 130 includes an authentication apparatus for authenticating the operator. For example, the authentication apparatus may be configured to carry out a face authentication by using an image obtained by the camera of the terminal apparatus 130 or may be configured to carry out a biological authentication by using biological information such as a fingerprint read by a reading sensor provided for the terminal apparatus 130.

After that, upon receipt of the instruction to start the medical examination from the terminal apparatus 130 (step S301: Yes), the identifying function 143a identifies the position of the terminal apparatus 130, by using a wireless communication technique such as WiFi (registered trademark) or Bluetooth (registered trademark) (step S302).

In addition, the identifying function 143a identifies a state of the operator, by using an image obtained by the camera 212 in the examination room 210 or by the camera 222 in the operation room 220 where the X-ray CT apparatus 110 is installed or an image obtained by the camera of the terminal apparatus 130 (step S303).

Furthermore, by using the authentication apparatus included in the terminal apparatus 130, the identifying function 143a identifies eligibility of the operator related to the operation to control the X-ray emissions and the operation to control the moving of the table 114 (step S304).

After that, on the basis of the position of the terminal apparatus 130 identified by the identifying function 143a, the judging function 143b judges whether or not the terminal apparatus 130 is positioned in a predetermined location other than the examination room 210 and the operation room 220 where the X-ray CT apparatus 110 is installed (step S305: Yes).

In this situation, for example, the predetermined location other than the examination room 210 and the operation room 220 denotes a location where, besides the examination room 210 and the operation room 220, it is permitted to perform, by using the terminal apparatus 130, the operation to control the X-ray emissions and the operation to control the moving of the table 114. For example, the predetermined location may be a location inside the hospital where the X-ray CT apparatus 110 is installed besides the examination room 210 and the operation room 220 or may be a location outside the hospital where the X-ray CT apparatus 110 is installed, such as the residence of a medical engineer or a medical doctor or another large-scale hospital.

Further, when the terminal apparatus 130 is positioned in the predetermined location (step S305: Yes), the judging function 143b judges whether or not the operator is an eligible person for the operation to control the X-ray emissions and the operation to control the moving of the table 114, on the basis of the eligibility of the operator identified by the identifying function 143a (step S306).

For example, when the operator is the medical engineer or the medical doctor who is in charge of the patient to be imaged or is a medical engineer or a medical doctor of a specific medical department, the judging function 143b determines that the operator is an eligible person for the operations.

Further, when the operator is an eligible person for the operations (step S306: Yes), the judging function 143b judges whether or not the operator is in a state of being able to view one of the X-ray CT apparatus 110 and the patient, on the basis of the state of the operator identified by the identifying function 143a (step S307).

For example, by judging the direction of the line of sight of the operator from an image obtained by the camera of the terminal apparatus 130, the judging function 143b determines that the operator is in the state of being able to view one of the X-ray CT apparatus 110 and the patient, when the line of sight of the operator is directed toward the terminal apparatus 130, while an image obtained by the camera 212 in the examination room 210 or by the camera 222 in the operation room 220 is displayed on the terminal apparatus 130.

After that, when the operator is in the state of being able to view one of the X-ray CT apparatus 110 and the patient (step S307: Yes), the judging function 143b determines that the safety is ensured about the operation to control the X-ray emissions and the operation to control the moving of the table 114 (step S308).

Subsequently, when the judging function 143b determines that the safety is ensured about the operation to control the X-ray emissions and the operation to control the moving of the table 114 (step S308), the controlling function 143c causes the terminal apparatus 130 to display the operation screen for receiving the operations from the operator, similarly to the first embodiment (step S309).

As a result, the terminal apparatus 130 is enabled to receive, from the operator, the operation to control the X-ray emissions and the operation to control the moving of the table 114.

As explained above, in the third embodiment, by employing the authentication apparatus included in the terminal apparatus 130, the identifying function 143a is configured to further identify the eligibility of the operator related to the operation to control the X-ray emissions and the operation to control the moving of the table 114. Further, when the terminal apparatus 130 is positioned in the predetermined location other than the rooms where the X-ray CT apparatus 110 is installed, while the operator is an eligible person for the operations and is in a state of being able to view one of the X-ray CT apparatus 110 and the patient, the judging function 143b is configured to determine that the safety is ensured about the operation to control the X-ray emissions and the operation to control the moving of the table 114.

With the configurations described above, even when the terminal apparatus 130 is used in the predetermined location other than the rooms where the X-ray CT apparatus 110 is installed, the terminal apparatus 130 is enabled to perform the certain operations that are performed by using the dedicated keypad 111 and operation panel 112. It is therefore possible to control the driving of the X-ray CT apparatus 110 with remote operations.

### Other Embodiments

Further, for example, in any of the embodiments described above, it is also acceptable to prompt the operator to perform an acknowledgment operation before enabling the terminal apparatus 130 to receive the operation to control the X-ray emissions and the operation to control the moving of the table 114.

More specifically, when the judging function 143b determines that the safety is ensured about the operation to control the X-ray emissions and the operation to control the moving of the table 114, the controlling function 143c may be configured, after issuing a notification via the terminal apparatus 130 indicating the capability to receive the operations, to control the terminal apparatus 130 so as to become able to receive the operation to control the X-ray emissions and the operation to control the moving of the table 114, at a point in time when the operator performs the acknowledgment operation to acknowledge the notification.

For example, when the judging function 143b determines that the safety is ensured about the operation to control the X-ray emissions and the operation to control the moving of the table 114, the controlling function 143c may be configured, at first, to cause the terminal apparatus 130 to display the notification message indicating the capability to receive the operations as well as an acknowledgment button. After that, at the point in time when the operator performs an operation to press the acknowledgment button, the controlling function 143c causes the terminal apparatus 130 to display the operation screen for receiving the operation to control the X-ray emissions and the operation to control the moving of the table 114.

With the configurations described above, it is possible to prevent the terminal apparatus 130 from displaying the operation screen for receiving the operation to control the X-ray emissions and the operation to control the moving of the table 114 when the operator does not have the intention (is not aware). It is therefore possible to prevent erroneous operations.

Further, for example, in any of the embodiments described above, it is also acceptable to issue an alert via the terminal apparatus 130, when the operator or someone else other than the operator is in a position having a possibility, from a safety viewpoint, of being affected by the X-ray emissions or the moving of the table 114.

More specifically, the identifying function 143a may be configured to identify states of the operator and someone else other than the operator who are present in the rooms where the X-ray CT apparatus 110 is installed.

For example, the identifying function 143a may be configured to identify the states of the operator and someone else, by using an image obtained by the camera 212 in the examination room 210 or by the camera 222 in the operation room 220 where the X-ray CT apparatus 110 is installed.

Further, the judging function 143b may be configured to further judge whether or not the operator or someone else is in a position having a possibility, from a safety viewpoint, of being affected by the X-ray emissions or the moving of the table 114, in the event that the operation to control the X-ray emissions or the operation to control the moving of the table 114 is performed.

For example, the judging function 143b may be configured to judge whether or not the operator or someone else is in a position to be exposed to the X-rays, in the event that the operation to control the X-ray emissions is performed. Also, for example, the judging function 143b may be configured to judge whether or not the operator or someone else is in a position with which the table 114 is to interfere, in the event that the operation to move the table 114 is performed.

Further, when the judging function 143b determines that the operator or someone else is in a position having the possibility of being affected by the X-ray emissions or the moving of the table 114, the controlling function 143c may be configured to issue the alert via the terminal apparatus 130.

For example, when the judging function 143b determines that the operator or someone else is in a position having the possibility of being affected by the X-ray emissions or the moving of the table 114, the controlling function 143c may be configured to issue an alert message detailing the affection or an alert sound via the terminal apparatus 130. On such occasion, the controlling function 143c may be configured to cause the terminal apparatus 130 to display the acknowledgment button together with the alert message or the alert sound and to further control the terminal apparatus 130 so as to become able to receive the operation to control the X-ray emissions and the operation to control the moving of the table 114, at the point in time when the operator performs the operation to press the acknowledgment button.

With the configurations described above, it is possible to more safely perform the operation to control the X-ray emissions and the operation to control the moving of the table 114, by using the terminal apparatus 130.

In the above embodiments, the example was explained in which the identifying function 143a is configured to identify the position of the terminal apparatus 130 and the state of the operator, whereas the judging function 143b is configured to judge whether or not the safety is ensured about the operation to control the X-ray emissions and the operation to control the moving of the table 114, on the basis of the position of the terminal apparatus 130 and the state of the operator; however, possible embodiments are not limited to this example.

For instance, the identifying function 143a may be configured to identify only the position of the terminal apparatus 130, whereas the judging function 143b may be configured to judge whether or not the safety is ensured about the operation to control the X-ray emissions and the operation to control the moving of the table 114, on the basis of only the position of the terminal apparatus 130.

Further, in the above embodiments, the example was explained in which, as the specific operations to control the driving of the X-ray CT apparatus 110, the terminal apparatus 130 is additionally enabled to perform the operation to control the X-ray emissions and the operation to control the moving of the table 114; however, possible embodiments are not limited to this example.

For instance, the operation which the terminal apparatus 130 is additionally enabled to perform may be an operation other than the operation to control the X-ray emissions and the operation to control the moving of the table 114, as long as the operation is a certain operation performed by using a dedicated operation machine directly linked to the X-ray CT apparatus 110 primarily from a safety viewpoint. Examples of such an operation include an operation to control moving of the gantry 113.

Further, in the above embodiments, the example was explained in which the operations related to the X-ray CT apparatus 110 are performed; however, possible embodiments are not limited to this example. For instance, it is possible to similarly apply any of the embodiments described above to an operation related to other medical machines, as long as the operation is a certain operation performed by using a dedicated operation machine directly linked to the medical machine primarily from a safety viewpoint. Examples of the other medical machines include X-ray diagnosis apparatuses, Magnetic Resonance Imaging (MRI) apparatuses, Positron Emission Tomography (PET) apparatuses, and Single Photon Emission Computed Tomography (SPECT) apparatuses.

Further, in the above embodiments, the example was explained in which the medical information processing system 100 includes the X-ray CT apparatus 110; however, the medical information processing system 100 may include a plurality of medical machines including the X-ray CT apparatus 110, an X-ray diagnosis apparatus, an MRI apparatus, a PET apparatus, a SPECT apparatus, and/or the like.

In that situation, for example, the judging function 143b may be configured to judge, with respect to each medical machine, whether or not safety is ensured about a specific operation to control driving of the medical machine, while the controlling function 143c may be configured to control the terminal apparatus 130 so as to become able to receive the operation from an operator for each medical machine. For example, with respect to each medical machine, the controlling function 143c may be configured to cause the terminal apparatus 130 to display an operation screen for receiving the specific operation.

Further, in the above embodiments, the example was explained in which the identifying unit, the judging unit, and the controlling unit of the present disclosure are realized by the identifying function, the judging function, and the controlling function of the processing circuitry, respectively; however, possible embodiments are not limited to this example. For instance, besides being realized by the identifying function, the judging function, and the controlling function described in the embodiments, functions of the identifying unit, the judging unit, and the controlling unit of the present disclosure may be realized by using hardware alone, software alone, or a combination of hardware and software.

Further, in the above embodiments, the example was explained in which the processing circuitry is realized by using the single processor; however, possible embodiments are not limited to this example. For instance, it is acceptable to structure the processing circuitry by combining together a plurality of independent processors, so that the processing functions are realized as a result of the processors executing the programs. Further, the processing functions of the processing circuitry may be realized as being distributed among or integrated into one or more pieces of processing circuitry, as appropriate. Further, the processing functions of the processing circuitry may be realized by using a combination of hardware such as circuitry and software. Furthermore, although the example was explained above in which the programs corresponding to the processing functions are stored in the single storage, possible embodiments are not limited to this example. For instance, another configuration is also acceptable in which the programs corresponding to the processing functions are stored in a plurality of storages in a distributed manner, so that the processing circuitry reads and executes the programs from the storages.

Further, the term “processor” used in the description of the above embodiments denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). In this situation, instead of having the programs saved in the storage, the programs may directly be incorporated in the circuitry of one or more processors. On such occasion, the one or more processors realize the functions by reading and executing the programs incorporated in the circuitry thereof. Further, the processors in the present embodiments do not each necessarily have to be structured as a single piece of circuitry. It is also acceptable to structure one processor by combining together a plurality of pieces of independent circuitry so as to realize the functions thereof.

In this situation, the programs executed by the one or more processors are provided as being incorporated, in advance, in a Read Only Memory (ROM), a storage, or the like. In other examples, the programs may be provided as being recorded in a non-transitory computer-readable storage medium such as a Compact Disk Read-Only Memory (CD-ROM), a Flexible Disk (FD), a Compact Disk Recordable (CD-R), a Digital Versatile Disk (DVD), or the like, in a file in a format that is installable or executable by these apparatuses. Further, the programs may be stored in a computer connected to a network such as the Internet so as to be provided or distributed as being downloaded via the network. For example, the programs are structured with modules including the processing functions described above. In the actual hardware, as a result of a CPU reading and executing the programs from a storage medium such as a ROM, the modules are loaded into a main storage apparatus and generated in the main storage apparatus.

Further, the constituent elements of the apparatuses depicted in the above embodiments and modification examples are based on functional concepts. Thus, it is not necessarily required to physically configure the constituent elements as indicated in the drawings. In other words, specific modes of distribution and integration of the apparatuses are not limited to those illustrated in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a CPU and a program analyzed and executed by the CPU or may be realized as hardware using wired logic.

Further, with regard to the processes explained in the above embodiments and modification examples, it is acceptable to manually perform all or a part of the processes described as being performed automatically. Conversely, by using a publicly-known method, it is also acceptable to automatically perform all or a part of the processes described as being performed manually. In addition, unless noted otherwise, it is acceptable to arbitrarily modify any of the processing procedures, the controlling procedures, specific names, and various information including various types of data and parameters that are presented in the above text and the drawings.

Furthermore, various types of data handled in the present disclosure are, typically, digital data.

According to at least one aspect of the embodiments described above, it is possible to conveniently perform the operations related to the medical machine.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A terminal controlling apparatus to control a terminal apparatus used by an operator of a medical machine, the terminal controlling apparatus comprising:
processing circuitry configured to
identify a position of the terminal apparatus;
judge, on a basis of the position of the terminal apparatus, whether or not safety is ensured about a specific operation to control driving of the medical machine; and
control, when it is determined that the safety of the operation is ensured, the terminal apparatus so as to become able to receive the operation from the operator.

2. The terminal controlling apparatus according to claim 1, wherein
the processing circuitry is configured to further identify a state of the operator, and
the processing circuitry is configured to judge whether or not the safety of the operation is ensured on a basis of the position of the terminal apparatus and the state of the operator.

3. The terminal controlling apparatus according to claim 2, wherein
the terminal apparatus is configured to be able to communicate with the terminal controlling apparatus by using a wireless communication technique,
the processing circuitry is configured to identify the position of the terminal apparatus by using the wireless communication technique, and
the processing circuitry determines that the safety of the operation is ensured, when the terminal apparatus is positioned inside a room in which the medical machine is installed, while the operator is in a state of being able to view one of the medical machine and an examined subject.

4. The terminal controlling apparatus according to claim 2, wherein
the medical machine includes a connection part to which the terminal apparatus is to be connected,
the processing circuitry is configured to identify the position of the terminal apparatus by detecting that the terminal apparatus is connected to the connection part, and
the processing circuitry determines that the safety of the operation is ensured, when the terminal apparatus is connected to the connection part, while the operator is facing the terminal apparatus straight on.

5. The terminal controlling apparatus according to claim 2, wherein
the processing circuitry is configured to further identify eligibility of the operator related to the operation, by using an authentication apparatus included in the terminal apparatus, and
the processing circuitry is configured to determine that the safety of the operation is ensured, when the terminal apparatus is positioned in a predetermined location other than a room in which the medical machine is installed, while the operator is an eligible person for the operation and is in a state of being able to view one of the medical machine and an examined subject.

6. The terminal controlling apparatus according to claim 1, wherein, the processing circuitry is configured to control the terminal apparatus so as to become able to receive the operation, when it is determined that the safety of the operation is ensured, after issuing, via the terminal apparatus, a notification indicating capability to receive the operation, at a point in time when the operator performs an acknowledgment operation to acknowledge the notification.

7. The terminal controlling apparatus according to claim 1, wherein
the processing circuitry is configured to identify states of the operator and at least one person other than the operator who are present in a room in which the medical machine is installed,
the processing circuitry is configured to further judge whether or not one of the operator and the at least one person is in a position that, when the operation is performed, has a possibility of being affected by the driving of the medical machine from a safety viewpoint, and
when it is determined that one of the operator and the at least one person is in the position that has the possibility of being affected, the processing circuitry is configured to issue an alert via the terminal apparatus.

8. The terminal controlling apparatus according to claim 1, wherein the processing circuitry is configured to identify a state of the operator, by using one or both of an image obtained by a camera provided inside a room in which the medical machine is installed and an image obtained by a camera of the terminal apparatus.

9. A terminal controlling method for controlling a terminal apparatus used by an operator of a medical machine, the terminal controlling method comprising:
identifying, by processing circuitry, a position of the terminal apparatus;
judging, by the processing circuitry, on a basis of the position of the terminal apparatus, whether or not safety is ensured about a specific operation to control driving of the medical machine; and
controlling, by the processing circuitry, when it is determined that the safety of the operation is ensured, the terminal apparatus so as to become able to receive the operation from the operator.

10. A non-transitory computer-readable storage medium having recorded thereon a plurality of computer-executable instructions for controlling a terminal apparatus used by an operator of a medical machine, wherein the plurality of instructions cause a computer to perform:
identifying a position of the terminal apparatus;
judging, on a basis of the position of the terminal apparatus, whether or not safety is ensured about a specific operation to control driving of the medical machine; and
controlling, when it is determined that the safety of the operation is ensured, the terminal apparatus so as to become able to receive the operation from the operator.
